(19) United States
(12) Patent Application Publication
(10) Pub. No.: US 2026/0248471 A1
(43) Pub. Date: Aug. 27, 2026

(54) X-RAY SHIELD FOR BREASTS

(71) Applicant: Patrick Allan Llewellyn, West Vancouver (CA)

(72) Inventor: Patrick Allan Llewellyn, West Vancouver (CA)

(21) Appl. No.: 19/549,427

(22) Filed: Feb. 25, 2026

Related U.S. Application Data
(60) Provisional application No. 63/762,876, filed on Feb. 25, 2025.

(51) Int. Cl. A61B 6/10

(52) U.S. Cl. CPC A61B 6/107

(57) ABSTRACT

An apparatus for shielding a breast region of a patient undergoing x-ray examination includes a central portion and first and second lateral portions. The central portion is suspended in front of the breast region. Each of the first and second lateral portions extend substantially perpendicularly from the central portion. The first lateral portion includes a first lateral edge, the second lateral portion includes a second lateral edge, and the first and second lateral edges are located apart from the central portion. The first and second lateral portions include material adapted to impede x-ray radiation. One of the first and second lateral edges is placed against a sternum of the patient during the x-ray examination.

### CROSS-REFERENCE TO RELATED APPLICATIONS

This application claims the benefit of United States Provisional Patent Application No. 63/762,876 filed Feb. 25, 2025, the contents of which are hereby incorporated by reference.

### TECHNICAL FIELD

The present disclosure relates to X-ray shielding, and in particular, to X-ray shielding for female breasts.

### BACKGROUND

Chest X-rays make up a significant percentage of diagnostic x-ray procedures. Most chest x-rays involve two views, a posterior-anterior (PA) view and a lateral view. The term “posterior-anterior” describes the path of the X-ray beam (i.e. from the posterior to the anterior of the patient). The X-ray beam is generated by an X-ray tube located behind the patient and the detector or film cassette is typically located against the patient’s anterior chest wall. Accordingly, the patient’s heart is nearest to the detector, and there is less magnification from the divergent X-ray beam. The X-ray beam may be made up of different energies around a set kilovoltage. The beam is not monochromatic and there are both higher and lower energy X-rays within the beam. Lower energy X-rays may be absorbed by the patient’s body and contribute to the patient’s radiation dose. The X-rays that are not absorbed by the body pass through the patient and create the image.

In a PA view, the X-ray beam passes from the X-ray tube through patient’s back and spine, the lungs and the heart, the anterior chest wall, and then finally the breasts before the beam contacts the detector. Much of the low-energy (and damaging) X-rays are absorbed by the body before they reach the breasts. In a lateral view, the anterior aspect of the entire X-ray beam passes directly through both breasts, as there is no filtering of the beam compared to the PA view. In addition, the lateral X-ray dose, measured in millisieverts (mSv), is on average up to four times the dose of a PA view. This is because the lateral width of the chest is greater than the PA width and therefore requires more energy to penetrate through the entire lateral width of the chest. For example, the average effective dose for a PA X-ray may be 0.02 mSv, while the average effective dose for a lateral X-ray may be 0.08 mSv.

The female breast is a radiosensitive organ and is the third most radiosensitive organ in the body (after the ovaries and the thyroid gland). Furthermore, the female breast is more radiosensitive in younger patients compared to post-menopausal females. The breasts cannot be effectively shielded in a PA view as that would obscure part of the image. However, as explained above, the dose for a PA view x-ray is typically lower than that for a lateral view x-ray.

There is therefore a need for an apparatus to provide shielding for a patient’s breasts and breast regions when undergoing lateral view x-rays.

### SUMMARY

An apparatus for shielding the breast regions of a patient when undergoing lateral view x-ray comprises a central portion and first and second lateral portions extending from the central portion. The central portion and the first and second lateral portions form a general U-shaped or C-shaped structure. An attachment member may be fixed on the central portion. The attachment member is adapted to removably attach the central portion to a stand.

One of the first and second lateral portions is positioned against the sternum of the patient. The central portion extends substantially parallel to the torso of the patient. The other of the first and second lateral portions extends over the lateral side of the breast of the patient (closest to the x-ray tube) and is effective in shielding both breasts from the x-ray beam.

In one aspect, an apparatus for shielding a breast region of a patient undergoing x-ray examination comprises a central portion and first and second lateral portions. The central portion is adapted to be suspended in front of the breast region. Each of the first and second lateral portions extend substantially perpendicularly from the central portion. The first lateral portion comprises a first lateral edge, the second lateral portion comprises a second lateral edge, and the first and second lateral edges are located apart from the central portion. The first and second lateral portions comprise material adapted to impede x-ray radiation. One of the first and second lateral edges is adapted to be placed against a sternum of the patient during the x-ray examination.

In another aspect, the first and second lateral portions comprise lead.

In yet another aspect, each of the first and second lateral portions comprises a lateral outer plate, a lateral inner plate, and a lateral medial plate. The lateral medial plate is located between the lateral outer plate and the lateral inner plate, and the lateral medial plate is made from lead.

In still another aspect, the lateral outer plate and the lateral inner plate are made from aluminum.

In still yet another aspect, one or both of the lateral outer plate and the lateral inner plate comprises a recessed portion. The lateral medial plate is located within the recessed portion.

In a further aspect, each of the lateral outer plate and the lateral inner plate comprises one or more lateral portion fastener openings located outside of the recessed portion. The one or more lateral portion fastener openings on the lateral outer plate are aligned with the one or more lateral portion fastener openings on the lateral inner plate.

In still a further aspect, the apparatus further comprises one or more fasteners adapted to pass through the one or more lateral portion fastener openings on the lateral outer plate and the lateral inner plate and adapted to secure the lateral outer plate to the lateral inner plate.

In another aspect, each of the lateral outer plate, the lateral inner plate, and the lateral medial plate comprises one or more lateral portion fastener openings. The one or more lateral portion fastener openings on the lateral outer plate are aligned with the one or more lateral portion fastener openings on the lateral inner plate and with the one or more lateral portion fastener openings on the lateral medial plate.

In yet another aspect, the apparatus further comprises one or more fasteners adapted to pass through the one or more lateral portion fastener openings on the lateral outer plate, the lateral inner plate, and the lateral medial plate and adapted to secure together the lateral outer plate, the lateral inner plate, and the lateral medial plate.

In a further aspect, the central portion comprises a central portion outer plate and a central portion inner plate.

In still a further aspect, the central portion outer plate is continuous with the lateral outer plates of the first and second lateral portions.

In still yet a further aspect, each of the central portion outer plate and the central portion inner plate comprises one or more central portion fastener openings. The one or more central portion fastener openings on the central portion outer plate are aligned with the one or more central portion fastener openings on the central portion inner plate.

In yet another aspect, the apparatus further comprises one or more fasteners adapted to pass through the one or more central portion fastener openings on the central portion outer plate and the central portion inner plate and adapted to secure the central portion outer plate to the central portion inner plate.

In still yet another aspect, the apparatus further comprises a padded member located on one or both of the first and second lateral portions proximate to the first and second lateral edges, respectively.

In a further aspect, the apparatus further comprises a stand adapted to attach to the central portion.

In still a further aspect, the stand comprises: a receiving member adapted to attach to the central portion, a frame, and a plurality of wheels secured to the frame. The receiving member is pivotably attached to the frame. The plurality of wheels is adapted to allow movement of the frame.

In another aspect, a method for shielding the breasts of a patient undergoing x-ray examination from an x-ray tube is provided. The method comprises providing an apparatus comprising a central portion and first and second lateral portions, wherein each of the first and second lateral portions extend substantially perpendicular from the central portion, wherein the first lateral portion comprises a first lateral edge, wherein the second lateral portion comprises a second lateral edge, wherein the first and second lateral edges are located apart from the central portion, and wherein the first and second lateral portions comprise material adapted to impede x-ray radiation; positioning the apparatus such that the central portion is suspended in front of the breasts; and placing one of the first and second lateral edges against a sternum of the patient such that the first and second lateral portions are located on either lateral sides of the breast closest to the x-ray tube.

In still another aspect, the apparatus further comprises a stand adapted to attach to the central portion. The stand comprises a plurality of wheels adapted to allow movement of the frame. The step of positioning the apparatus such that the central portion is suspended in front of the breasts comprises moving the apparatus on the wheels.

The foregoing was intended as a summary only and of only some of the aspects. It was not intended to define the limits or requirements of the present disclosure. Other aspects will be appreciated by reference to the detailed description set out below.

### BRIEF DESCRIPTION OF DRAWINGS

The embodiments of the present disclosure will be described by reference to the drawings thereof, in which:

FIG. 1 depicts the apparatus in accordance with one embodiment;

FIG. 2 depicts the apparatus in accordance with another embodiment;

FIG. 3 is another view of the apparatus of FIG. 2;

FIG. 4 is a partial exploded view of the apparatus in accordance with one embodiment;

FIG. 5 is a partial exploded view of the apparatus in accordance with another embodiment;

FIG. 6 depicts the apparatus in use with a patient; and

FIG. 7 is a partial sectional top view of the apparatus in use with a patient.

### DETAILED DESCRIPTION

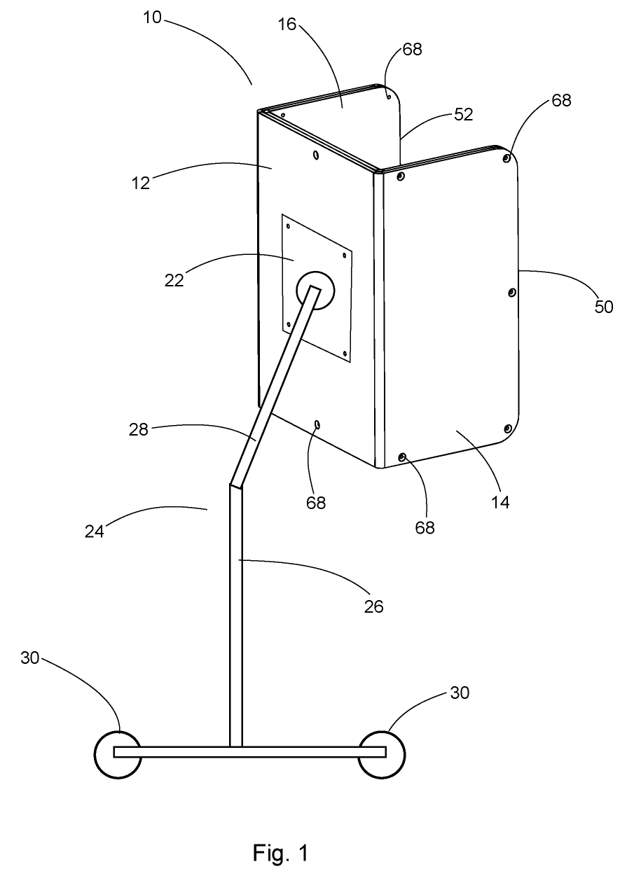

Referring to FIGS. 1 to 3, an apparatus 10 for shielding the breasts or breast regions of a patient 2 undergoing x-ray examination comprises a central portion 12 and first and second lateral portions 14, 16, with the first and second lateral portions 14, 16 extending from the central portion 12. In one embodiment, the central portion 12 and the first and second lateral portions 14, 16 form a general U-shaped or C-shaped structure when viewed from above. For example, the central portion 12 may comprise first and second central portion edges 18, 20 from which the first and second lateral portions 14, 16, respectively, extend. In other embodiments, the first and second lateral portions 14, 16 may extend from the central portion 12 offset from the first and second central portion edges 18, 20.

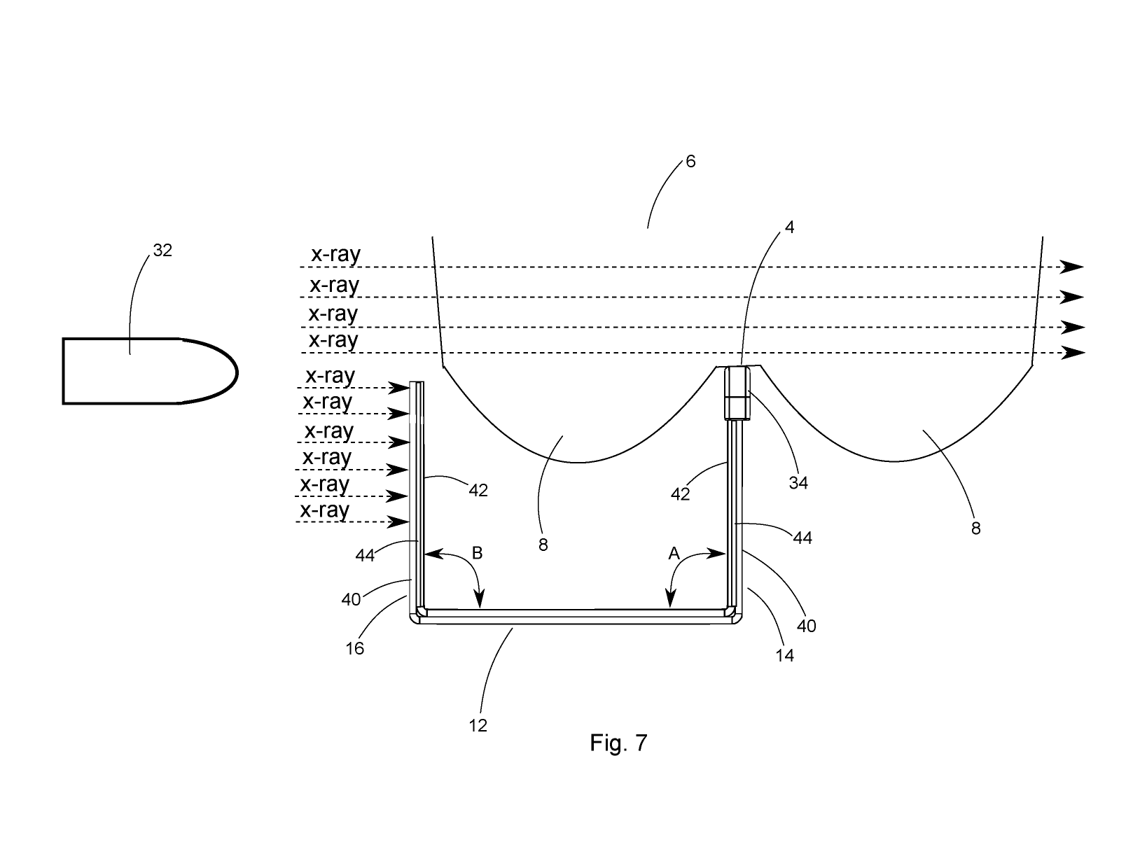

In some embodiments, the first and second lateral portions 14, 16 extend substantially perpendicularly from the central portion 12. Referring to FIG. 7, an angle A between the first lateral portion 14 and the central portion 12 may be approximately 90°, and an angle B between the second lateral portion 16 and the central portion 12 may be approximately 90°. In other embodiments, the angle A may be between 85° and 95°, and the angle B may be between 85° and 95°. In still other embodiments, instead of the first and second lateral portions 14, 16 extending from the central portion 12 at an angle, one or both of the first and second lateral portions 14, 16 may extend from the central portion 12 through a curvature.

The first lateral portion 14 comprises a first lateral edge 50, and the second lateral portion 16 comprises a second lateral edge 52. The first lateral edge 50 and the second lateral edge 52 may define the ends of the general U-shaped or C-shaped structure formed by the apparatus 10. The first lateral portion 14 further comprises a first upper edge 54 and a first lower edge 56, and the second lateral portion further comprises a second upper edge 58 and a second lower edge 60.

In one embodiment, one or more of the central portion 12 and the first and second lateral portions 14, 16 may be generally planar. In other embodiments, one or more of the central portion 12 and/or the first and second lateral portions 14, 16 may be curved. In the embodiment shown in FIG. 1, all of the central portion 12 and the first and second lateral portions 14, 16 are generally planar. In addition, in the embodiment shown in FIG. 1, the first and second lateral portions 14, 16 extend substantially perpendicularly from the central portion 12.

In some embodiments, the first and second lateral portions 14, 16 may be generally rectangular in shape. The first and second lateral portions 14, 16 may be substantially similar in size. The central portion 12 may also be generally rectangular in shape.

One or more of the central portion 12 and the first and second lateral portions 14, 16 may comprise material that is able to block or impede x-ray radiation. In one embodiment, at least the first and second lateral portions 14, 16 are made from material that is able to block or impede x-ray radiation. For example, the first and second lateral portions 14, 16 may be made from lead or some other suitable material. In some embodiments, only a portion of the first and/or second lateral portions 14, 16 may be made from material that is able to block or impede x-ray radiation.

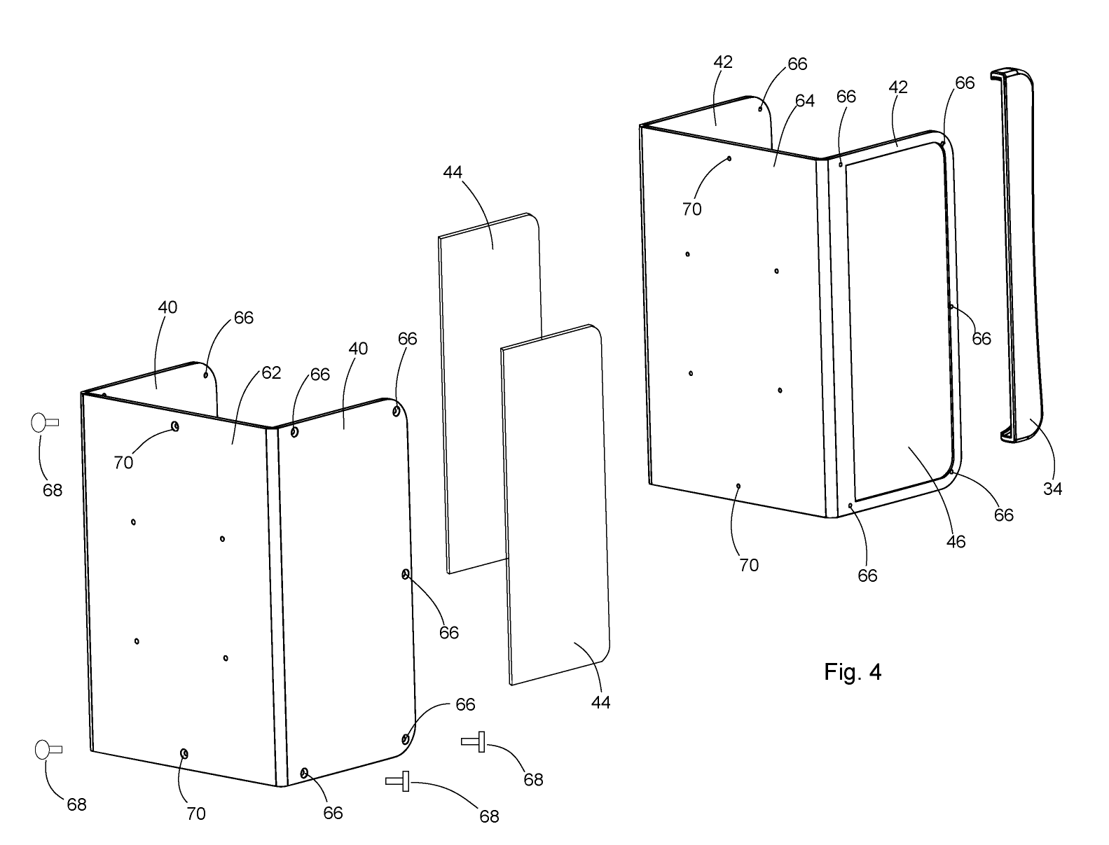

Referring to FIG. 4, which is an exploded view, in some embodiments, each of the first and second lateral portions 14, 16 may comprise a lateral portion outer plate 40, a lateral portion inner plate 42, and a lateral portion medial plate 44 sandwiched between the lateral portion outer plate 40 and the lateral portion inner plate 42. The lateral portion medial plate 44 may be made from lead with a thickness equal to or greater than 0.5mm. In other embodiments, the lateral portion medial plate 44 may be made from a material that has at least 0.5 mm lead equivalent. The lateral portion outer plate 40 and the lateral portion inner plate 42 may be made from aluminum or some other suitable material.

In some embodiments, referring to FIG. 4, at least one of the lateral portion outer plate 40 and the lateral portion inner plate 42 may comprise a recessed portion 46 within which the lateral portion medial plate 44 may occupy when the lateral portion medial plate 44 is sandwiched between the lateral portion outer plate 40 and the lateral portion inner plate 42. For example, in the embodiment shown in FIG. 4, the lateral portion inner plate 42 comprises the recessed portion 46; however, it is understand that the recessed portion 46 may be present in either one or both of the lateral portion outer plate 40 and the lateral portion inner plate 42.

In other embodiments, the lateral portion medial plate 44 may extend to the first lateral edge 50 or the second lateral edge 52. In some embodiments, the lateral portion medial plate 44 may also extend to the first upper edge 54 and/or the first lower edge 56. The lateral portion medial plate 44 may also extend to the second upper edge 58 and/or the second lower edge 60.

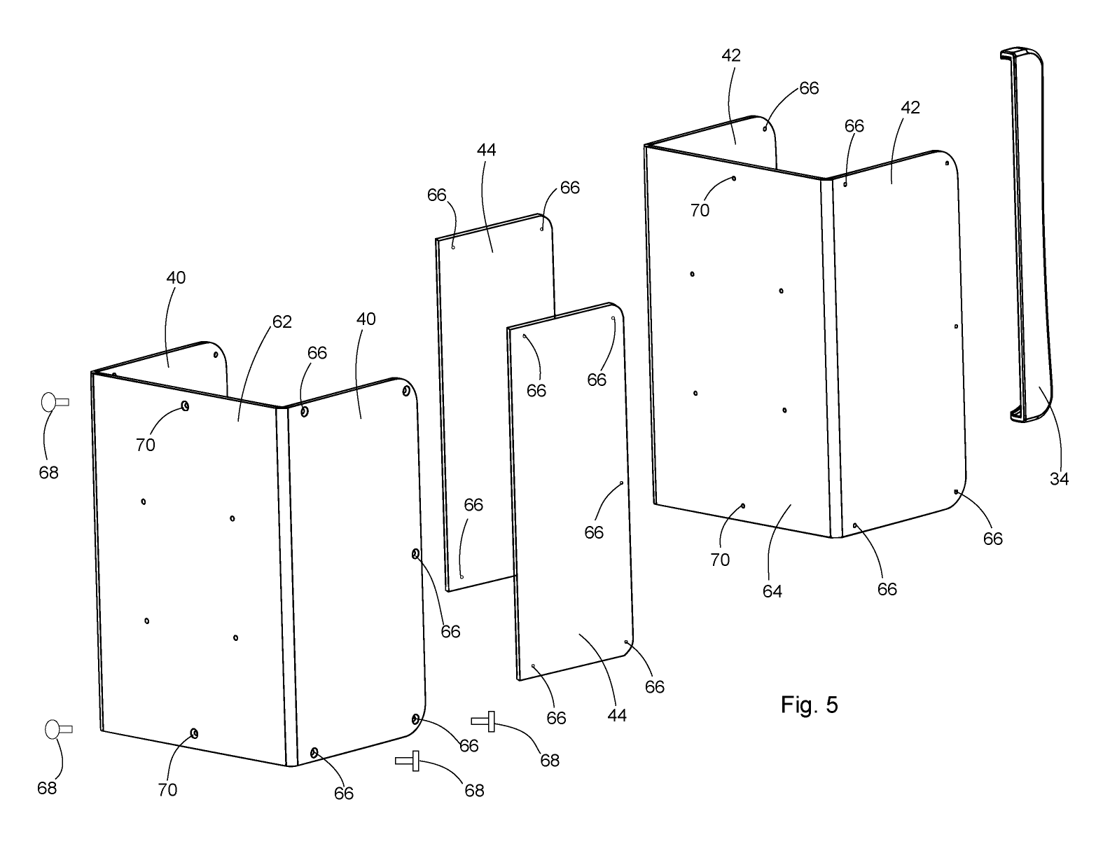

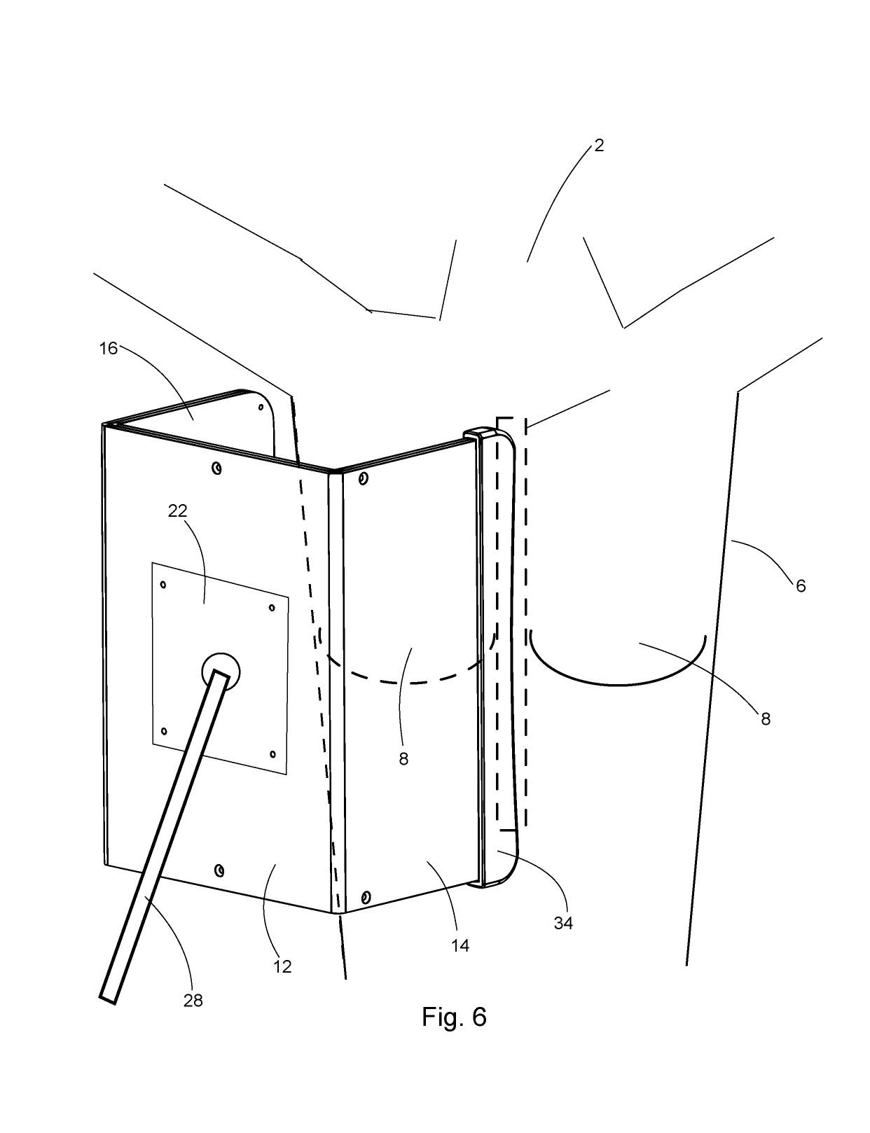

In other embodiments, referring to FIG. 5, which is another exploded view, the recessed portion 44 is not present in either of the lateral portion outer plate 40 or the lateral portion inner plate 42.

In some embodiments, referring to FIGS. 4 and 5, the central portion 12 may comprise a central portion outer plate 62 and a central portion inner plate 64. In one embodiment, the central portion outer plate 62 may be continuous with the lateral portion outer plates 40 of the first lateral portion 14 and the second lateral portion 16. In other words, the central portion outer plate 62 may be formed from bending a single sheet of material at the first and second central portion edges 18, 20. Furthermore, in some embodiments, the central portion inner plate 64 may be continuous with the lateral portion inner plates 42 of the first lateral portion 14 and the second lateral portion 16. For example, the central portion inner plate 64 may be formed from bending a single sheet of material at the first and second central portion edges 18, 20.

The lateral portion outer plate 40 and the lateral portion inner plate 42 may comprise one or more lateral portion fastener openings 66 that are adapted to receive fasteners 68 for securing the lateral portion outer plate 40 to the lateral portion inner plate 42. The lateral portion fastener openings 66 on the lateral portion outer plate 40 may be aligned with the lateral portion fastener openings 66 on the lateral portion inner plate 42 so as to allow the fasteners 68 to pass therethrough. The fasteners 68 may be screws, bolts, rivets, or any other suitable fasteners.

In some embodiments, the lateral portion fastener openings 66 are located on the lateral portion outer plate 40 and the lateral portion inner plate 42 such that they lie outside of the recessed portion 46. In such embodiments, the fasteners 68 would not interfere or intrude upon the lateral portion medial plate 44.

In other embodiments, where the recessed portion 46 is not present (such as depicted in FIG. 5), the lateral portion medial plate 44 may also comprise one or more of the lateral portion fastener openings 66. The lateral portion fastener openings 66 on the lateral portion outer plate 40, the lateral portion medial plate 44, and the lateral portion inner plate 42 may be aligned so as to allow the fasteners 68 to pass therethrough.

In some embodiments, the central portion outer plate 62 and the central portion inner plate 64 may comprise one or more central portion fastener openings 70 that are adapted to receive the fasteners 68 for securing the central portion outer plate 62 to the central portion inner plate 64. The central portion fastener openings 70 on the central portion outer plate 62 may be aligned with the central portion fastener openings 70 on the central portion inner plate 64 so as to allow the fasteners 68 to pass therethrough.

Referring back to FIG. 1, the apparatus 10 further comprises an attachment member 22 that may be fixed on the central portion 12. The attachment member 22 may take the form of a plate that is secured to the central portion 12. The attachment member 22 is adapted to removably detach from a stand 24. In some embodiments, the stand 24 may be part of the apparatus 10, while in other embodiments, the stand 24 may be separate from the apparatus 10.

The stand 24 may comprise a frame 26 and a receiving member 28 for engaging with the attachment member 22. The receiving member 28 may be pivotably attached to one or both of the attachment member 22 and the frame 26 to allow for vertical adjustment of the central portion 12. The stand 24 may further comprise a plurality of wheels 30 secured to or mounted to the frame 26 to allow the stand 24 to be moved about.

Referring to FIGS. 6 and 7, the apparatus 10 may be used with the patient 2 by positioning the apparatus 10 such that one of the first lateral edge 50 or the second lateral edge 52 is placed against the sternum 4 of the patient 2. If necessary, a height of the central portion 12 may be adjusted by pivoting the receiving member 28 with respect to the frame 26 and/or the central portion 12 until the central portion 12 is located in front of the breasts 8. If the first lateral edge 50 is placed against the sternum 4, the first lateral portion 14 may be angled such that it extends substantially perpendicularly to the torso 6 of the patient 2. As a result, the central portion 12 may extend substantially parallel to the torso 6, in a direction away from the sternum 4. The second lateral portion 16 may extend over the lateral sides of one of the breasts 8 of the patient 2. Therefore, when a lateral view x-ray of the patient 2 is taken (such as from an x-ray tube 32), the x-ray beam is substantially blocked from passing through the breasts 8 by the second lateral portion 16.

Similarly, if the second lateral edge 52 is placed against the sternum 4, the second lateral portion 16 may be angled such that it extends substantially perpendicularly to the torso 6. As a result, the central portion 12 may extend substantially parallel to the torso 6, in a direction away from the sternum 4. The first lateral portion 14 may extend over the lateral sides of one of the breasts 8 of the patient 2. Therefore, when a lateral view x-ray of the patient 2 is taken, the x-ray beam is substantially blocked from passing through the breasts 8 by the first lateral portion 14.

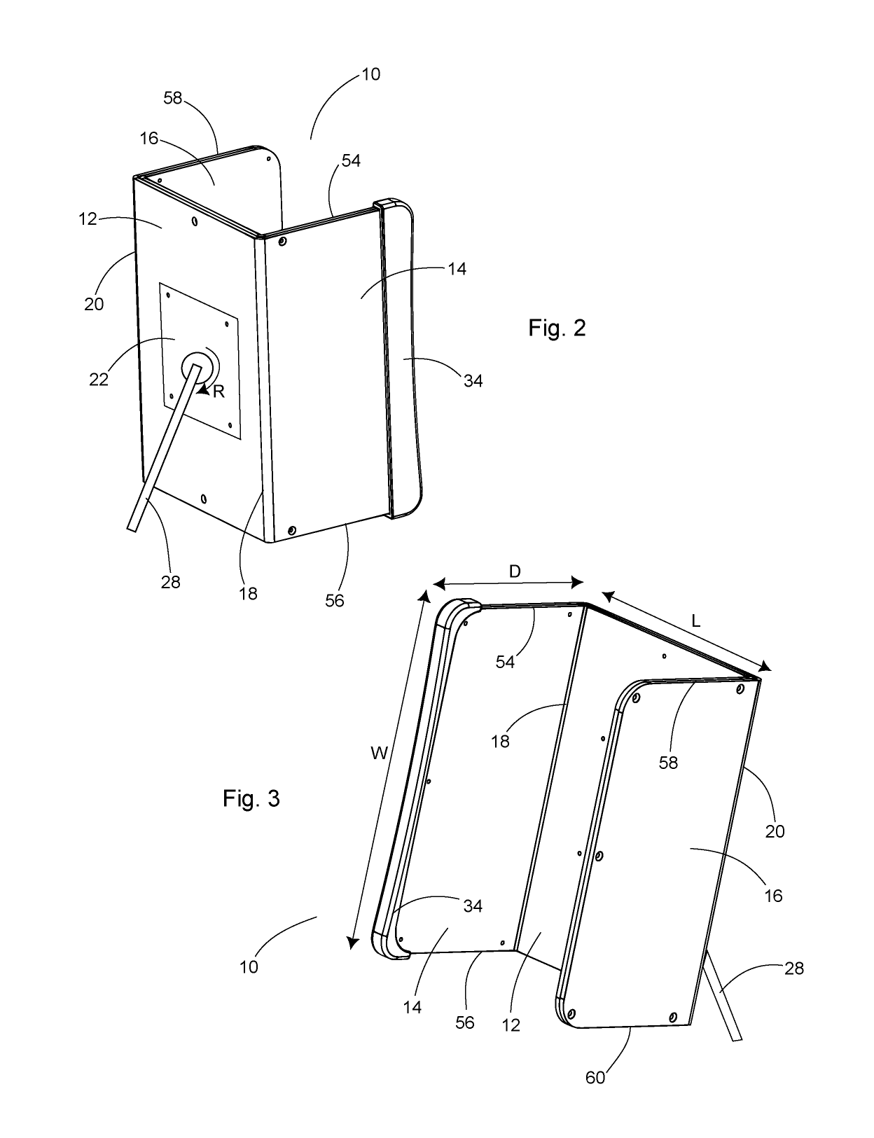

In some embodiments, the apparatus 10 may comprise one or more padded members 34 attached to one or both of the first lateral portion 14 and the second lateral portion 16 proximate to the first lateral edge 50 and the second lateral edge 52, respectively. In the embodiment shown in FIGS. 2 and 3, the padded member 34 is attached to first lateral portion 14 proximate to the first lateral edge 50. It is understood that one of the padded members 34 may also be attached to the second lateral portion 16 proximate to the second lateral edge 52. The padded member 34 may provide some cushioning of the first lateral edge 50 and/or the second lateral edge 52 against the sternum 4, depending on whether the padded member 34 is located on one or both of the first and second lateral portions 14, 16.

In some embodiments, the padded member 34 may be located on only one of the first lateral edge 50 or the second lateral edge 52 (e.g. on only the first lateral edge 50). In such embodiments, the central portion 12 and the first and second lateral portions 14, 16 may be rotated 180° (for example, in direction R, as shown in FIG. 2) so that the second lateral portion 16 may be used to substantially block the x-ray beam from either lateral side of the breasts 8.

In embodiments where the apparatus 10 does not comprise the stand 24, the apparatus 10 may be otherwise held or suspended in front of the breasts 8 by other means, such as the apparatus 10 being suspended from a ceiling or the like.

Referring to FIG. 3, in some embodiments, the central portion 12 and the first and second lateral portions 14, 16 are substantially rectangular in shape. It is understood that other shapes for the central portion 12 and the first and second lateral portions 14, 16 are possible. The apparatus 10 may have a length L of up to 30 cm, a width W of up to 20 cm, and a depth D of up to 20 cm.

The apparatus 10 may also assist in keeping the patient 2 substantially upright while the x-ray is being taken. By placing the sternum 4 against the first or second lateral edges 50, 52, the patient 2 may be prevented from leaning to one side while the x-ray is taken.

It will be appreciated by those skilled in the art that certain embodiments have been described in some detail but that certain modifications may be practiced without departing from the principles of the invention.

1. An apparatus for shielding a breast region of a patient undergoing x-ray examination, the apparatus comprising:
a central portion, wherein the central portion is adapted to be suspended in front of the breast region; and
first and second lateral portions, wherein each of the first and second lateral portions extend substantially perpendicularly from the central portion, wherein the first lateral portion comprises a first lateral edge, wherein the second lateral portion comprises a second lateral edge, and wherein the first and second lateral edges are located apart from the central portion;
wherein the first and second lateral portions comprise material adapted to impede x-ray radiation; and
wherein one of the first and second lateral edges is adapted to be placed against a sternum of the patient during the x-ray examination.

2. The apparatus of claim 1, wherein the first and second lateral portions comprise lead.

3. The apparatus of claim 2, wherein each of the first and second lateral portions comprises:
a lateral outer plate;
a lateral inner plate; and
a lateral medial plate located between the lateral outer plate and the lateral inner plate, wherein the lateral medial plate is made from lead.

4. The apparatus of claim 3, wherein the lateral outer plate and the lateral inner plate are made from aluminum.

5. The apparatus of claim 3, wherein one or both of the lateral outer plate and the lateral inner plate comprises a recessed portion, and wherein the lateral medial plate is located within the recessed portion.

6. The apparatus of claim 5, wherein each of the lateral outer plate and the lateral inner plate comprises one or more lateral portion fastener openings located outside of the recessed portion, wherein the one or more lateral portion fastener openings on the lateral outer plate are aligned with the one or more lateral portion fastener openings on the lateral inner plate.

7. The apparatus of claim 6, further comprising one or more fasteners adapted to pass through the one or more lateral portion fastener openings on the lateral outer plate and the lateral inner plate and adapted to secure the lateral outer plate to the lateral inner plate.

8. The apparatus of claim 3, wherein each of the lateral outer plate, the lateral inner plate, and the lateral medial plate comprises one or more lateral portion fastener openings, wherein the one or more lateral portion fastener openings on the lateral outer plate are aligned with the one or more lateral portion fastener openings on the lateral inner plate and with the one or more lateral portion fastener openings on the lateral medial plate.

9. The apparatus of claim 8, further comprising one or more fasteners adapted to pass through the one or more lateral portion fastener openings on the lateral outer plate, the lateral inner plate, and the lateral medial plate and adapted to secure together the lateral outer plate, the lateral inner plate, and the lateral medial plate.

10. The apparatus of claim 3, wherein the central portion comprises a central portion outer plate and a central portion inner plate.

11. The apparatus of claim 10, wherein the central portion outer plate is continuous with the lateral outer plates of the first and second lateral portions.

12. The apparatus of claim 10, wherein each of the central portion outer plate and the central portion inner plate comprises one or more central portion fastener openings, wherein the one or more central portion fastener openings on the central portion outer plate are aligned with the one or more central portion fastener openings on the central portion inner plate.

13. The apparatus of claim 12, further comprising one or more fasteners adapted to pass through the one or more central portion fastener openings on the central portion outer plate and the central portion inner plate and adapted to secure the central portion outer plate to the central portion inner plate.

14. The apparatus of claim 1, further comprising a padded member located on one or both of the first and second lateral portions proximate to the first and second lateral edges, respectively.

15. The apparatus of claim 1, further comprising a stand adapted to attach to the central portion.

16. The apparatus of claim 15, wherein the stand comprises:
a receiving member adapted to attach to the central portion;
a frame, wherein the receiving member is pivotably attached to the frame; and
a plurality of wheels secured to the frame, the plurality of wheels adapted to allow movement of the frame.

17. A method for shielding the breasts of a patient undergoing x-ray examination from an x-ray tube, the method comprising:
providing an apparatus comprising a central portion and first and second lateral portions, wherein each of the first and second lateral portions extend substantially perpendicular from the central portion, wherein the first lateral portion comprises a first lateral edge, wherein the second lateral portion comprises a second lateral edge, wherein the first and second lateral edges are located apart from the central portion, and wherein the first and second lateral portions comprise material adapted to impede x-ray radiation;
positioning the apparatus such that the central portion is suspended in front of the breasts; and
placing one of the first and second lateral edges against a sternum of the patient such that the first and second lateral portions are located on either lateral sides of the breast closest to the x-ray tube.

18. The method of claim 17, wherein the apparatus further comprises a stand adapted to attach to the central portion, wherein the stand comprises a plurality of wheels adapted to allow movement of the frame, and wherein the step of positioning the apparatus such that the central portion is suspended in front of the breasts comprises moving the apparatus on the wheels.
